(19) United States
(12) Patent Application Publication
(10) Pub. No.: US 2022/0409122 A1
(43) Pub. Date: Dec. 29, 2022

(54) Artificial Intelligence Assisted Medical Diagnosis Method For Sepsis And System Thereof

(71) Applicant: China Medical University, Taichung City (TW)

(72) Inventors: Der-Yang Cho, Taichung City (TW); Kai-Cheng Hsu, Taichung City (TW); Ya-Lun Wu, Taichung City (TW); Ding-Hong Xu, Taichung City (TW)

(73) Assignee: China Medical University, Taichung City (TW)

(21) Appl. No.: 17/472,899

(22) Filed: Sep. 13, 2021

(30) Foreign Application Priority Data
Jun. 28, 2021 (TW) 110123616

(51) Int. Cl. A61B 5/00; G16H 50/20; G16H 50/30; G16H 10/60; G06K 9/62

(52) U.S. Cl. CPC A61B 5/412; G16H 50/20; G16H 50/30; G16H 10/60; G06K 9/6262

(57) ABSTRACT

An artificial intelligence assisted medical diagnosis method for a sepsis is proposed. A database reading step is performed to read a sepsis database and at least one database to be tested of a storing unit. The sepsis database includes a plurality of sepsis data, and the at least one database to be tested includes a plurality of data to be tested. A data table creating step is performed to create a sepsis data table according to the sepsis data, and create a data table to be tested according to the data to be tested. A model training step is performed to train the sepsis data table according to a K-fold cross-validation and a machine learning algorithm to generate a sepsis diagnosis model. A sepsis predicting step is performed to input the data table to be tested into the sepsis diagnosis model to calculate a sepsis prediction result.

### RELATED APPLICATIONS

This application claims priority to Taiwan Application Serial Number 110123616, filed Jun. 28, 2021, which is herein incorporated by reference.

### BACKGROUND

### Technical Field

The present disclosure relates to a medical diagnosis method and a system thereof. More particularly, the present disclosure relates to an artificial intelligence assisted medical diagnosis method for a sepsis and a system thereof.

### Description of Related Art

Sepsis is a common and life-threatening syndrome, which causes extremely high morbidity and mortality. In addition, sepsis is not easy to be diagnosed in time, so that patients cannot receive proper treatment immediately, which lead to Severe Sepsis or Septic Shock.

The conventional diagnosis method of sepsis requires medical personnel to repeatedly confirm the medical information of patient, and thus it is easy to miss the prime time for treatment. For example, the conventional diagnosis method of sepsis is to observe the physiologically values of vital signs and test report of a subject, or Electronic Medical Record (EMR). Medical personnel diagnose whether the subject is a septic patient according to the physiologically values. In addition to the physiological values, there have been more the related values of special bacteria or biomarkers as a standard for measuring sepsis, recently.

The diagnosis of sepsis is based on the evidence of infection plus symptoms of systemic inflammation, and the initial sepsis sometimes lacks specific symptoms for the diagnosis. When clear symptoms appear, the patient may have entered the stage of septic shock or Multiple Organ Dysfunction Syndrome (MODS).

In view of this, how to establish a medical diagnosis method for the sepsis and a system thereof that can assist and speed up the treatment of subjects by medical personnel for the problems existing in the conventional diagnosis method of sepsis is indeed highly anticipated by the public and become the goal and the direction of relevant industry efforts.

### SUMMARY

According to one aspect of the present disclosure, an artificial intelligence assisted medical diagnosis method for a sepsis includes performing a database reading step, a data table creating step, a model training step and a sepsis predicting step. The database reading step is performed to drive a processing unit to read a sepsis database and at least one database to be tested of a storing unit. The sepsis database includes a plurality of sepsis data, and the at least one database to be tested includes a plurality of data to be tested. The data table creating step is performed to drive the processing unit to create a sepsis data table according to the sepsis data and create a data table to be tested according to the data to be tested. The model training step is performed to drive the processing unit to train the sepsis data table according to a K-fold cross-validation and a machine learning algorithm to generate a sepsis diagnosis model. The sepsis predicting step is performed to drive the processing unit to input the data table to be tested into the sepsis diagnosis model to calculate a sepsis prediction result.

According to another aspect of the present disclosure, an artificial intelligence assisted medical diagnosis system for a sepsis includes a storing unit and a processing unit. The storing unit is configured to access a sepsis database, at least one database to be tested, a K-fold cross-validation and a machine learning algorithm. The sepsis database includes a plurality of sepsis data, and the at least one database to be tested includes a plurality of data to be tested. The processing unit is connected to the storing unit and configured to implement an artificial intelligence assisted medical diagnosis method for the sepsis including performing a data table creating step, a model training step and a sepsis predicting step. The data table creating step is performed to create a sepsis data table according to the sepsis data and create a data table to be tested according to the data to be tested. The model training step is performed to train the sepsis data table according to the K-fold cross-validation and the machine learning algorithm to generate a sepsis diagnosis model. The sepsis predicting step is performed to input the data table to be tested into the sepsis diagnosis model to calculate a sepsis prediction result.

### BRIEF DESCRIPTION OF THE DRAWINGS

The present disclosure can be more fully understood by reading the following detailed description of the embodiment, with reference made to the accompanying drawings as follows:

FIG. 1 shows a flow chart of an artificial intelligence assisted medical diagnosis method for a sepsis according to a first embodiment of the present disclosure.

FIG. 2 shows a flow chart of an artificial intelligence assisted medical diagnosis method for the sepsis according to a second embodiment of the present disclosure.

FIG. 3A shows a schematic view of a database reading step of the artificial intelligence assisted medical diagnosis method for the sepsis of FIG. 2.

FIG. 3B shows a schematic view of a patient basic data, a patient vital sign data, a patient blood test data, a subject basic data, a subject vital sign data and a subject blood test data of FIG. 3A.

FIG. 4 shows a schematic view of a Receiver Operating Characteristic Curve (ROC Curve) of a sepsis diagnosis model of FIG. 2.

FIG. 5 shows a block diagram of an artificial intelligence assisted medical diagnosis system for the sepsis according to a third embodiment of the present disclosure.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to the other element, or it can be indirectly connected to the other element, that is, intervening elements may be present. In contrast, when an element is referred to as be “directly connected to” another element, there are no intervening elements present. In addition, the terms first, second, third, etc. are used herein to describe various elements or components, these elements or components should not be limited by these terms. Consequently, a first element or component discussed below could be termed a second element or component.

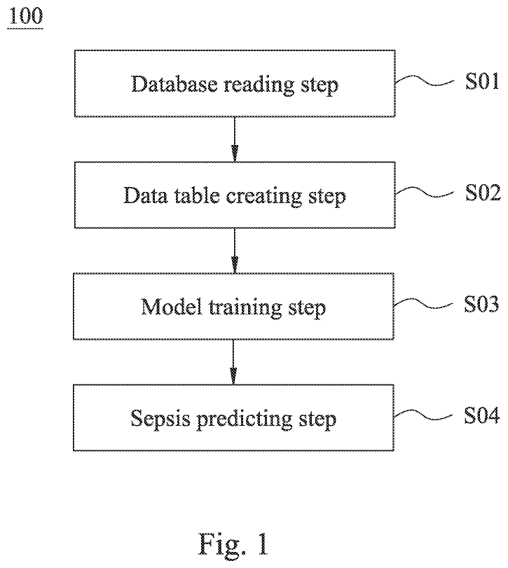

Please refer to FIG. 1. FIG. 1 shows a flow chart of an artificial intelligence assisted medical diagnosis method 100 for a sepsis according to a first embodiment of the present disclosure. In FIG. 1, the artificial intelligence assisted medical diagnosis method 100 for the sepsis includes performing a database reading step S01, a data table creating step S02, a model training step S03 and a sepsis predicting step S04.

The database reading step S01 is performed to drive a processing unit to read a sepsis database and at least one database to be tested of a storing unit. The sepsis database includes a plurality of sepsis data, and the at least one database to be tested includes a plurality of data to be tested. The data table creating step S02 is performed to drive the processing unit to create a sepsis data table according to the sepsis data and create a data table to be tested according to the data to be tested. The model training step S03 is performed to drive the processing unit to train the sepsis data table according to a K-fold cross-validation (K-fold CV) and a machine learning algorithm to generate a sepsis diagnosis model. The sepsis predicting step S04 is performed to drive the processing unit to input the data table to be tested into the sepsis diagnosis model to calculate a sepsis prediction result.

Therefore, the artificial intelligence assisted medical diagnosis method 100 for the sepsis of the present disclosure takes advantage of the sepsis diagnosis model to predict the risk of the sepsis early, and can predict the onset of sepsis within 12 hours before clinical recognition, so that medical personnel can perform the follow-up medical treatment of patients earlier so as to significantly shorten the hospital stay for critical patients and reduce the mortality rate.

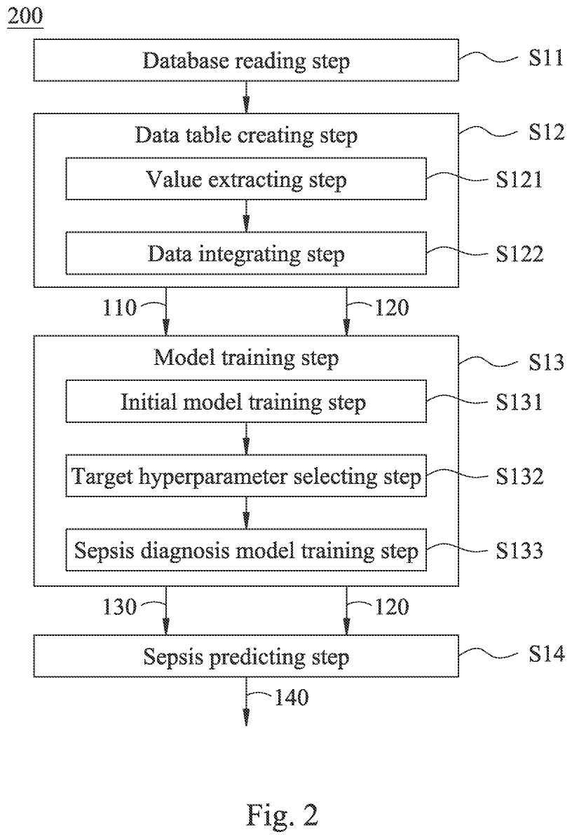

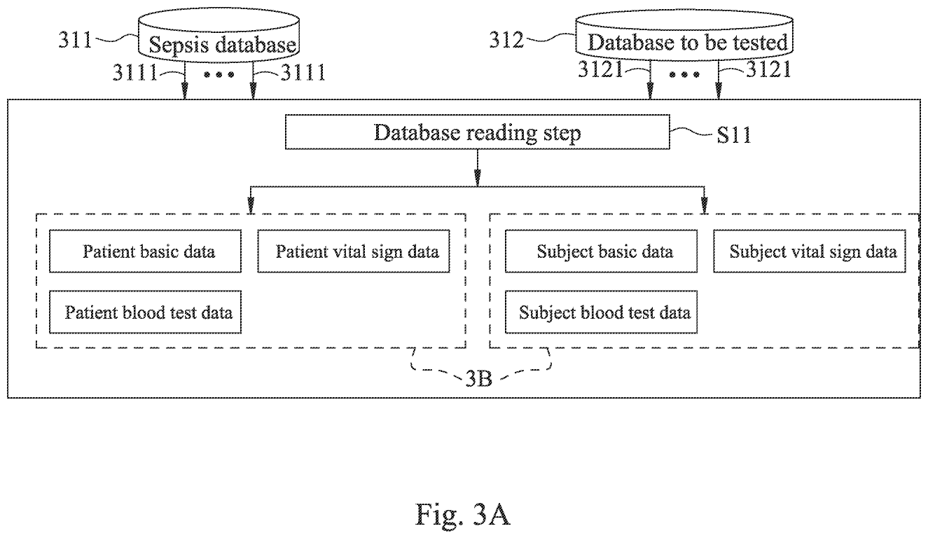

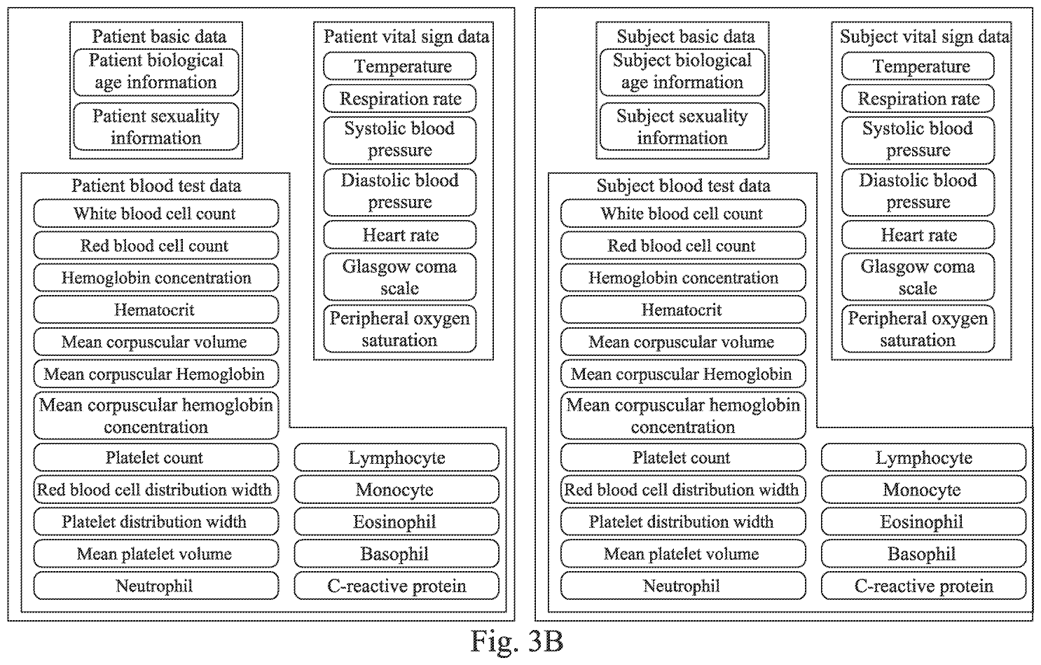

Please refer to FIGS. 2, 3A and 3B. FIG. 2 shows a flow chart of an artificial intelligence assisted medical diagnosis method 200 for a sepsis according to a second embodiment of the present disclosure. FIG. 3A shows a schematic view of a database reading step S11 of the artificial intelligence assisted medical diagnosis method 200 for the sepsis of FIG. 2. FIG. 3B shows a schematic view of a patient basic data, a patient vital sign data, a patient blood test data, a subject basic data, a subject vital sign data and a subject blood test data of FIG. 3A. In FIGS. 2, 3A and 3B, the artificial intelligence assisted medical diagnosis method 200 for the sepsis is mainly configured to input a data table 120 to be tested corresponding to a subject into a sepsis diagnosis model 130 to calculate a sepsis prediction result 140, and includes performing a database reading step S11, a data table creating step S12, a model training step S13 and a sepsis predicting step S14.

The database reading step S11 is performed to drive a processing unit to read a sepsis database 311 and a database to be tested 312 of a storing unit. The sepsis database 311 includes a plurality of sepsis data 3111, and the database to be tested 312 includes a plurality of data to be tested 3121.

In detail, the sepsis data 3111 can be a patient basic data, a patient vital sign data and a patient blood test data, respectively. The patient basic data can include a patient biological age information and a patient sexuality information. The patient vital sign data can include a temperature, a respiration rate, a Systolic Blood Pressure (SBP), a Diastolic Blood Pressure (DBP), a heart rate, a Glasgow Coma Scale (GCS) and a peripheral oxygen saturation (SpO₂). The patient blood test data can include a white blood cell count, a red blood cell count, a hemoglobin concentration, a hematocrit, a mean corpuscular volume, a mean corpuscular hemoglobin, a mean corpuscular hemoglobin concentration, a platelet count, a red blood cell distribution width, a platelet distribution width, a mean platelet volume, a neutrophil, a lymphocyte, a monocyte, an eosinophil, a basophil and a C-reactive protein, but the present disclosure is not limited thereto.

On the other hand, the data to be tested 3121 can be a subject basic data, a subject vital sign data and a subject blood test data, respectively. The subject basic data can include a subject biological age information and a subject sexuality information. The subject vital sign data can include a temperature, a respiration rate, a Systolic Blood Pressure (SBP), a Diastolic Blood Pressure (DBP), a heart rate, a Glasgow Coma Scale (GCS) and a peripheral oxygen saturation (SpO₂) corresponding to the subject. The subject blood test data can include a white blood cell count, a red blood cell count, a hemoglobin concentration, a hematocrit, a mean corpuscular volume, a mean corpuscular hemoglobin, a mean corpuscular hemoglobin concentration, a platelet count, a red blood cell distribution width, a platelet distribution width, a mean platelet volume, a neutrophil, a lymphocyte, a monocyte, an eosinophil, a basophil and a C-reactive protein corresponding to the subject, but the present disclosure is not limited thereto.

The data table creating step S12 is performed to drive the processing unit to create a sepsis data table 110 according to the sepsis data 3111 and create a data table 120 to be tested according to the data to be tested 3121. Specifically, the data table creating step S12 can include a value extracting step S121 and a data integrating step S122.

The value extracting step S121 is performed to drive the processing unit to extract a maximum value, a minimum value and a latest value (i.e., the most recent value for a sepsis patient) of each of the temperature, the respiration rate, the SBP, the DBP, the heart rate, the GCS and the SpO₂ of the patient vital sign data.

The data integrating step S122 is performed to drive the processing unit to integrate the maximum values, the minimum values and the latest values of the patient vital sign data, the patient basic data and the patient blood test data to generate the sepsis data table 110; similarly, the processing unit integrates the maximum values, the minimum values and the latest values of the subject vital sign data, the subject basic data and the subject blood test data to generate the data table 120 to be tested. It can be seen that the sepsis data table 110 of the present disclosure collects various physiological data of the sepsis patient after a period of time as the features for modeling the follow-up machine learning model, and the period of time is referred to as a feature window. Each of the sepsis patients in the feature window may not have the same inspection frequency for each of the features. The present disclosure unifies the number of the features used for modeling in order to preserve the numerical changes that may be of clinical concern, so that a large amount of the data of each of the features are simplified into three features (that is, the aforementioned maximum value, minimum value and latest value), and then use the three features to build the follow-up machine learning model.

The model training step S13 is performed to drive the processing unit to train the sepsis data table 110 according to a K-fold cross-validation (K-fold CV) and a machine learning algorithm to generate a sepsis diagnosis model 130. In particular, the model training step S13 can include an initial model training step S131, a target hyperparameter selecting step S132 and a sepsis diagnosis model training step S133.

The initial model training step S131 is performed to drive the processing unit to cut the sepsis data table 110 into K data sets according to the K-fold CV. The K data sets include K−1 training sets and a validation set, and then the processing unit trains the K−1 training sets according to a plurality of initial hyperparameters and the machine learning algorithm to generate a plurality of initial models corresponding to each of the initial hyperparameters.

In detail, the K-fold CV, the machine learning algorithm and the initial hyperparameters corresponding to the machine learning algorithm have been stored in the aforementioned storing unit. The variable K of the present disclosure can be 5, and the machine learning algorithm is an eXtreme Gradient Boosting (XGBoost), but the present disclosure is not limited thereto.

In response to determine that K=5, the sepsis data table 110 is cut into 5 data sets, and the 5 data sets are a first data set, a second data set, a third data set, a fourth data set and a fifth data set, respectively. In a first verification, the first data set, the second data set, the third data set and the fourth data set are used as the training sets, and the fifth data set is used as the validation set. The processing unit trains the 4 training sets according to one of the initial hyperparameters and the machine learning algorithm to generate an initial model corresponding to the one of the initial hyperparameters.

In a second verification, the first data set, the second data set, the third data set and the fifth data set are used as the training sets, and the fourth data set is used as the validation set. The processing unit also trains the 4 training sets according to the one of the initial hyperparameters and the machine learning algorithm to generate another initial model corresponding to the one of the initial hyperparameters, and so on, repeating the verification 5 times to generate 5 initial models corresponding to the one of the initial hyperparameters. In other words, the K-fold CV uses the K−1 data sets as the training sets, and uses the remaining data set as the validation set. Then, the data set that has not been the validation set is selected as the validation set in the next verification. The previously verified validation set is changed back to the training set, and changed iteratively until each of the data sets has been the validation set, so that K times of the verifications are performed, and K initial models are generated. It should be noted that the processing unit will perform a verification corresponding to another initial hyperparameter and repeat the verification 5 times to generate another 5 initial models corresponding to the another initial hyperparameter.

Then, the target hyperparameter selecting step S132 is performed to drive the processing unit to calculate the initial models through the validation set to generate a plurality of mean area under curves corresponding to the initial models, and then compare the mean area under curves to select a target hyperparameter from the initial hyperparameters. In detail, the processing unit uses the fifth data set (i.e., the validation set) to calculate the initial model to generate an Area Under Curve (AUC) during the aforementioned first verification; similarly, the processing unit uses different validation sets to perform calculations on the initial models corresponding to different validation sets to generate another AUC. The processing unit averages the AUCs corresponding to the one of the initial hyperparameters to generate one of the mean area under curves, and then selects the initial hyperparameter corresponding to the mean area under curve having the higher value as the target hyperparameter.

The sepsis diagnosis model training step S133 is performed to drive the processing unit to retrain the sepsis data table 110 according to the target hyperparameter and the machine learning algorithm to generate the sepsis diagnosis model 130. The sepsis predicting step S14 is performed to drive the processing unit to input the data table 120 to be tested into the sepsis diagnosis model 130 to calculate a sepsis prediction result 140.

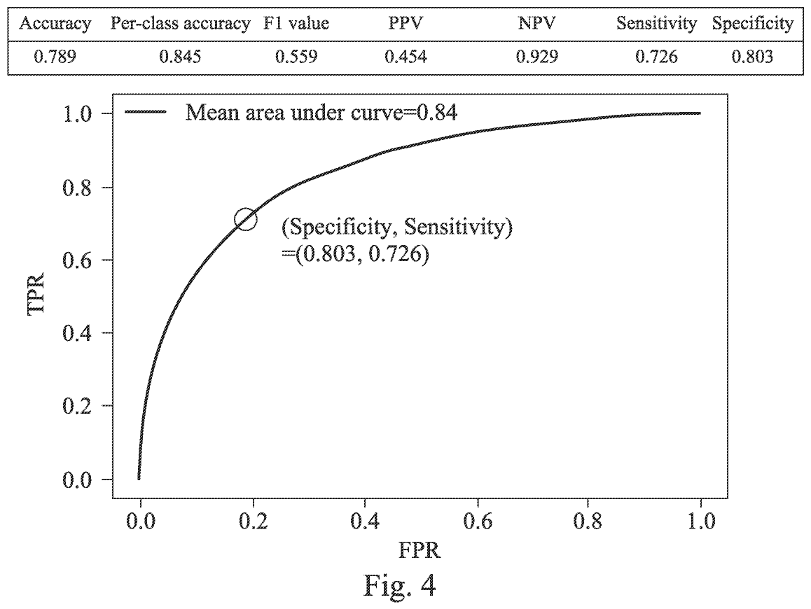

Please refer to FIGS. 2, 3A, 3B and 4. FIG. 4 shows a schematic view of a Receiver Operating Characteristic Curve (ROC Curve) of the sepsis diagnosis model 130 of FIG. 2. The present disclosure uses the K-fold CV to ensure that each of the data sets of the sepsis data table 110 participates in the training and the verification so as to reduce the deviation of the sepsis diagnosis model 130 and improve the accuracy of the diagnosis of sepsis. In FIG. 4, the mean area under curve of the ROC Curve of the sepsis diagnosis model 130 can be 0.84, and a cut-off value of the sepsis diagnosis model 130 can be 0.5. In response to determine that the sepsis prediction result 140 is greater than or equal to the cut-off value, the subject is judged to be the sepsis patient. In addition, an accuracy corresponding to the cut-off value can be 0.789, a per-class accuracy can be 0.845, a F1 value (that is, a harmonic mean of a precision and a recall) can be 0.559, a Positive Predictive Value (PPV) can be 0.454, a Negative Predictive Value (NPV) can be 0.929, a sensitivity can be 0.726, and a specificity can be 0.803, but the present disclosure is not limited thereto.

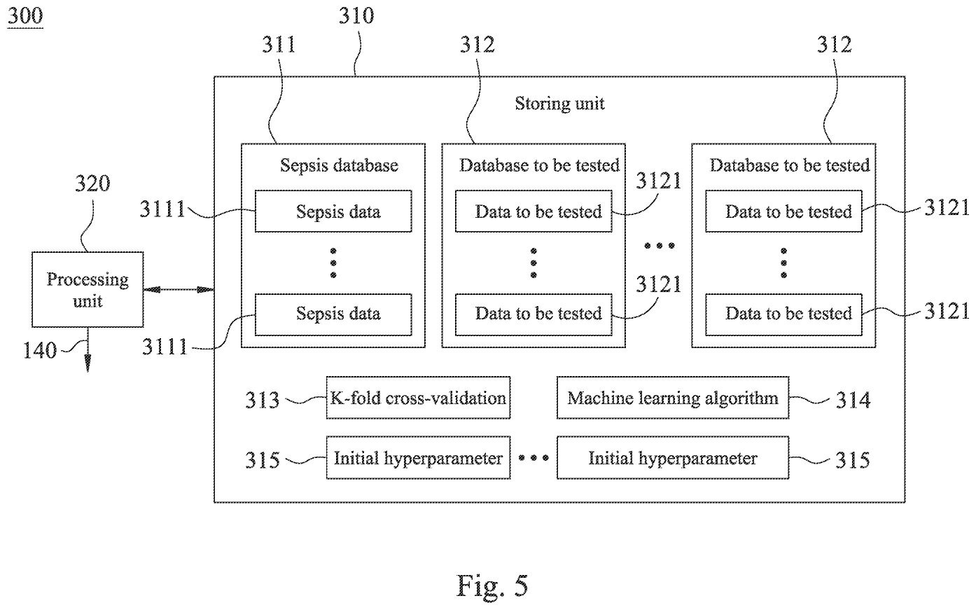

Please refer to FIGS. 2-4 and 5. FIG. 5 shows a block diagram of an artificial intelligence assisted medical diagnosis system 300 for the sepsis according to a third embodiment of the present disclosure. In FIGS. 2-5, the artificial intelligence assisted medical diagnosis system 300 for the sepsis is mainly configured to input the data table 120 to be tested corresponding to the subject into the sepsis diagnosis model 130 to calculate the sepsis prediction result 140, and includes a storing unit 310 and a processing unit 320.

The storing unit 310 is configured to access the sepsis database 311, a plurality of databases to be tested 312, a K-fold CV 313, a machine learning algorithm 314 and a plurality of initial hyperparameters 315. The sepsis database 311 includes a plurality of sepsis data 3111, and each of the databases to be tested 312 includes a plurality of data to be tested 3121. In particular, the sepsis data 3111 is multiple clinical data and various test reports of the sepsis patient. Each of the databases to be tested 312 corresponds to different subjects, and the data to be tested 3121 is multiple clinical data and various test reports of one of the subjects. The storing unit 310 can be a Hospital Information System (HIS) or a cloud server.

The processing unit 320 is signally connected to the storing unit 310 and configured to implement including performing a data table creating step S12, a model training step S13 and a sepsis predicting step S14. The data table creating step S12 is performed to create the sepsis data table 110 according to the sepsis data 3111 and create the data table 120 to be tested according to the data to be tested 3121. The model training step S13 is performed to train the sepsis data table 110 according to the K-fold CV 313 and the machine learning algorithm 314 to generate the sepsis diagnosis model 130. The sepsis predicting step S14 is performed to input the data table 120 to be tested into the sepsis diagnosis model 130 to calculate the sepsis prediction result 140. In addition, the processing unit 320 can be a wearable device, or a Micro Processing Unit (MPU), a Central Processing Unit (CPU), an image processor or other electronic processors of an Intensive Care Unit (ICU) electronic equipment, but the present disclosure is not limited thereto.

Therefore, the artificial intelligence assisted medical diagnosis system 300 for the sepsis of the present disclosure collects the sepsis data 3111 of the sepsis patient, and then trains the sepsis diagnosis model 130 through the K-fold CV 313 and the machine learning algorithm 314 to predict the risk of the sepsis for the subject. In addition to reducing the loadings on many medical personnel, the sepsis patients or the subjects can be assisted by medical personnel faster.

In summary, the present disclosure has the following advantages. First, taking advantage of the sepsis diagnosis model to predict the risk of the sepsis early, so that medical personnel can perform the follow-up medical treatment of patients earlier so as to significantly shorten the hospital stay for critical patients and reduce the mortality rate. Second, it is favorable for using the K-fold CV to reduce the deviation of the sepsis diagnosis model and improve the accuracy of the diagnosis of sepsis. Third, the trained sepsis diagnosis model can be combined with the wearable device or applied to the intelligence of the ICU, and the trained sepsis diagnosis model can screen more accurately and provide the real-time monitoring for patients having the risk of sepsis infection.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An artificial intelligence assisted medical diagnosis method for a sepsis, comprising:
performing a database reading step to drive a processing unit to read a sepsis database and at least one database to be tested of a storing unit, wherein the sepsis database comprises a plurality of sepsis data, and the at least one database to be tested comprises a plurality of data to be tested;
performing a data table creating step to drive the processing unit to create a sepsis data table according to the sepsis data and create a data table to be tested according to the data to be tested;
performing a model training step to drive the processing unit to train the sepsis data table according to a K-fold cross-validation and a machine learning algorithm to generate a sepsis diagnosis model; and
performing a sepsis predicting step to drive the processing unit to input the data table to be tested into the sepsis diagnosis model to calculate a sepsis prediction result.

2. The artificial intelligence assisted medical diagnosis method for the sepsis of claim 1, wherein the sepsis data are a patient basic data, a patient vital sign data and a patient blood test data.

3. The artificial intelligence assisted medical diagnosis method for the sepsis of claim 2, wherein the data table creating step comprises:
performing a value extracting step to drive the processing unit to extract a maximum value, a minimum value and a latest value of the patient vital sign data; and
performing a data integrating step to drive the processing unit to integrate the maximum value, the minimum value, the latest value, the patient basic data and the patient blood test data to generate the sepsis data table.

4. The artificial intelligence assisted medical diagnosis method for the sepsis of claim 2, wherein the patient basic data comprises a patient biological age information and a patient sexuality information.

5. The artificial intelligence assisted medical diagnosis method for the sepsis of claim 2, wherein the patient vital sign data comprises a temperature, a respiration rate, a Systolic Blood Pressure (SBP), a Diastolic Blood Pressure (DBP), a heart rate, a Glasgow Coma Scale (GCS) and a peripheral oxygen saturation (SpO₂).

6. The artificial intelligence assisted medical diagnosis method for the sepsis of claim 2, wherein the patient blood test data comprises a white blood cell count, a red blood cell count, a hemoglobin concentration, a hematocrit, a mean corpuscular volume, a mean corpuscular hemoglobin, a mean corpuscular hemoglobin concentration, a platelet count, a red blood cell distribution width, a platelet distribution width, a mean platelet volume, a neutrophil, a lymphocyte, a monocyte, an eosinophil, a basophil and a C-reactive protein.

7. The artificial intelligence assisted medical diagnosis method for the sepsis of claim 1, wherein the model training step comprises:
performing an initial model training step to drive the processing unit to cut the sepsis data table into K data sets according to the K-fold cross-validation, wherein the K data sets comprise K−1 training sets and a validation set, and then the processing unit trains the K−1 training sets according to a plurality of initial hyperparameters and the machine learning algorithm to generate a plurality of initial models corresponding to each of the initial hyperparameters;
performing a target hyperparameter selecting step to drive the processing unit to calculate the initial models through the validation set to generate a plurality of mean area under curves corresponding to the initial models, and then compare the mean area under curves to select a target hyperparameter from the initial hyperparameters; and
performing a sepsis diagnosis model training step to drive the processing unit to retrain the sepsis data table according to the target hyperparameter and the machine learning algorithm to generate the sepsis diagnosis model.

8. The artificial intelligence assisted medical diagnosis method for the sepsis of claim 1, wherein the machine learning algorithm is an eXtreme Gradient Boosting (XGBoost).

9. An artificial intelligence assisted medical diagnosis system for a sepsis, comprising:
a storing unit configured to access a sepsis database, at least one database to be tested, a K-fold cross-validation and a machine learning algorithm, wherein the sepsis database comprises a plurality of sepsis data, and the at least one database to be tested comprises a plurality of data to be tested; and
a processing unit connected to the storing unit, wherein the processing unit is configured to implement an artificial intelligence assisted medical diagnosis method for the sepsis comprising:
performing a data table creating step to create a sepsis data table according to the sepsis data and create a data table to be tested according to the data to be tested;
performing a model training step to train the sepsis data table according to the K-fold cross-validation and the machine learning algorithm to generate a sepsis diagnosis model; and
performing a sepsis predicting step to input the data table to be tested into the sepsis diagnosis model to calculate a sepsis prediction result.

10. The artificial intelligence assisted medical diagnosis system for the sepsis of claim 9, wherein the sepsis data are a patient basic data, a patient vital sign data and a patient blood test data.

11. The artificial intelligence assisted medical diagnosis system for the sepsis of claim 10, wherein the data table creating step comprises:
performing a value extracting step to extract a maximum value, a minimum value and a latest value of the patient vital sign data; and
performing a data integrating step to integrate the maximum value, the minimum value, the latest value, the patient basic data and the patient blood test data to generate the sepsis data table.

12. The artificial intelligence assisted medical diagnosis system for the sepsis of claim 10, wherein the patient basic data comprises a patient biological age information and a patient sexuality information.

13. The artificial intelligence assisted medical diagnosis system for the sepsis of claim 10, wherein the patient vital sign data comprises a temperature, a respiration rate, a Systolic Blood Pressure (SBP), a Diastolic Blood Pressure (DBP), a heart rate, a Glasgow Coma Scale (GCS) and a peripheral oxygen saturation (SpO₂).

14. The artificial intelligence assisted medical diagnosis system for the sepsis of claim 10, wherein the patient blood test data comprises a white blood cell count, a red blood cell count, a hemoglobin concentration, a hematocrit, a mean corpuscular volume, a mean corpuscular hemoglobin, a mean corpuscular hemoglobin concentration, a platelet count, a red blood cell distribution width, a platelet distribution width, a mean platelet volume, a neutrophil, a lymphocyte, a monocyte, an eosinophil, a basophil and a C-reactive protein.

15. The artificial intelligence assisted medical diagnosis system for the sepsis of claim 9, wherein the model training step comprises:
performing an initial model training step to drive the processing unit to cut the sepsis data table into K data sets according to the K-fold cross-validation, wherein the K data sets comprise K−1 training sets and a validation set, and then the processing unit trains the K−1 training sets according to a plurality of initial hyperparameters and the machine learning algorithm to generate a plurality of initial models corresponding to each of the initial hyperparameters;
performing a target hyperparameter selecting step to drive the processing unit to calculate the initial models through the validation set to generate a plurality of mean area under curves corresponding to the initial models, and then compare the mean area under curves to select a target hyperparameter from the initial hyperparameters; and
performing a sepsis diagnosis model training step to drive the processing unit to retrain the sepsis data table according to the target hyperparameter and the machine learning algorithm to generate the sepsis diagnosis model.

16. The artificial intelligence assisted medical diagnosis system for the sepsis of claim 9, wherein the machine learning algorithm is an eXtreme Gradient Boosting (XGBoost).
